(19) United States
(12) Patent Application Publication
(10) Pub. No.: US 2008/0108835 A1
(43) Pub. Date: May 8, 2008

(54) Method For Purifying (1S)-3-Methylamino-1-(2-Thienyl)-1-Propanol

(76) Inventors: Rainer Sturmer, Rodersheim-Gronau (DE); Jurgen Dauwel, Heidelberg (DE); Maria Kesseler, Mannheim (DE); Brigitte Achatz, Mannheim (DE); Michael Breuer, Darmstadt (DE)

Correspondence Address: CONNOLLY BOVE LODGE & HUTZ, LLP, P O BOX 2207, WILMINGTON, DE, 19899, US

(21) Appl. No.: 11/814,253

(22) PCT Filed: Dec. 31, 2005
(86) PCT No.: PCT/EP05/14161

§ 371 Date: Jul. 18, 2007

(30) Foreign Application Priority Data
Jan. 5, 2005 (DE) 10 2005 000 867.4

(51) Int. Cl. C07D 333/20

(52) U.S. Cl. 549/75

(57) ABSTRACT

The present invention relates to a method for purifying (1S)-3-METHYLAMINO-1-(2-THIENYL)-1-PROPANOL by distilling a reaction mixture which comprises between 25 and 99% by weight of (IS)-3-METHYLAMINO-1-(2-THIENYL)-1-PROPANOL.

The present invention relates to a method for purifying (1S)-3-methylamino-1-(2-thienyl)-1-propanol.

### PRIOR ART

(1S)-3-methylamino-1-(2-thienyl)-1-propanol is an important intermediate for producing drugs, in particular Duloxetin®, a pharmaceutical active compound from EliLilly & Co, USA,

The preparation of (1S)-3-methylamino-1-(2-thienyl)-1-propanol has been disclosed. A common preparation method, which is described in detail in DE 103 48 479 A1, involves reacting thiophene with 3-chloropropionyl chloride in the presence of AlCl₃, subsequently enantioselectively reducing the keto group to a hydroxyl group and directly aminating to give (1S)-3-methylamino-1-(2-thienyl)-1-propanol. The (1S)-3-methylamino-1-(2-thienyl)-1-propanol is subsequently purified by recrystallization, preferably from methyl tert-butyl ether.

The preparation of (1S)-3-methylamino-1-(2-thienyl)-1-propanol is also described in WO 2004/031168. In this case, too, the (1S)-3-methylamino-1-(2-thienyl)-1-propanol is purified by crystallization from methyl tert-butyl ether.

The preparation of (1S)-3-methylamino-1-(2-thienyl)-1-propanol is also described in WO 2004/005307. In this case, too, the (1S)-3-methylamino-1-(2-thienyl)-1-propanol is purified by crystallization from methyl tert-butyl ether.

The above-described methods for purifying (1S)-3-methylamino-1-(2-thienyl)-1-propanol are characterized by significant yield losses.

### OBJECT

It was the object of the present invention to make available a method for purifying (1S)-3-methylamino-1-(2-thienyl)-1-propanol which supplies this valuable product both in high chemical and enantiomeric purity and in high yield and which should also be possible to implement on an industrial scale at costs which are justifiable.

### DETAILED DESCRIPTION OF THE INVENTION

We have found that this object is achieved by means of a method for purifying (1S)-3-methylamino-1-(2-thienyl)-1-propanol by distilling a reaction mixture which comprises between 25 and 99% by weight of (1S)-3-methylamino-1-(2-thienyl)-1-propanol.

The term “reaction mixture” is intended to have the following meaning: the reaction mixture can be the unpurified mixture derived from the reaction for preparing (1S)-3-methylamino-1-(2-thienyl)-1-propanol, that is, for example, the reaction mixture composed of methylamine and (1S)-3-chloro-1-(2-thienyl)-1-propanol. However, the reaction mixture can, depending on the route for preparing (1S)-3-methylamino-1-(2-thienyl)-1-propanol, be a mixture composed of other coreactants. However, the term “reaction mixture” also encompasses the purified or partially purified reaction product (1S)-3-methylamino-1-(2-thienyl)-1-propanol. A solution of (1S)-3-methylamino-1-(2-thienyl)-1-propanol in one or more solvents is also intended to be understood as being “a reaction mixture which comprises ((1S)-3-methylamino-1-(2-thienyl)-1-propanol”.

As already mentioned above, one route for preparing (1S)-3-methylamino-1-(2-thienyl)-1-propanol is disclosed in detail in DE 103 48 479A1, which, with regard to the preparation of (1S)-3-methylamino-1-(2-thienyl)-1-propanol, is expressly incorporated herein by reference.

However, the method according to the invention can also be very successfully used for (1S)-3-methylamino-1-(2-thienyl)-1-propanol which has been prepared in another manner, for example as described in WO 2004/031168 and in WO 2004/005307, which publications are likewise expressly incorporated herein by reference. It is also possible to conceive of other routes for preparing (1S)-3-methylamino-1-(2-thienyl)-1-propanol, for example enantioselectively reducing the keto group to the hydroxyl function by enzymic means or reversing the reaction sequence described in DE 103 48479, i.e. directly aminating the 3-chloro-1-(2-thienyl)-1-propanone and subsequently reducing to give (1S)-3-methylamino-1-(2-thienyl)-1-propanol. The method according to the invention is suitable for all these preparation methods as well as for other methods, which are not specifically mentioned here, for preparing (1S)-3-methylamino-1-(2-thienyl)-1-propanol.

The reaction mixture usually comprises the valuable product (1S)-3-methylamino-1-(2-thienyl)-1-propanol in a quantity of from 25 to 99% by weight, preferably of from 40 to 98% by weight, particularly preferably in a quantity of more than 50% by weight.

If the (1S)-3-methylamino-1-(2-thienyl)-1-propanol is present in the reaction mixture in a quantity of less than 25% by weight, it is usually advisable to carry out a concentration step prior to the distillation, for example by removing solvents or concentrating the (1S)-3-methylamino-1-(2-thienyl)-1-propanol by means of extraction.

The distillation can be carried out in a large number of forms known to the skilled person, as described, for example, in manuals of chemical technology, such as Ullmann or Winnacker-Küchler.

Particularly preferred forms of the method according to the invention are what are termed short-path or molecular distillations, in which the distillation is carried out using paths between evaporation and condensation spaces which are as short and rectilinear as possible.

Commercially available molecular distillation appliances, as are offered for sale in the specialist trade, are also particularly suitable.

The method according to the invention makes it possible to isolate (1S)-3-methylamino-1-(2-thienyl)-1-propanol in unexpectedly high chemical yield, and at the same time at a high degree of purity, in one single operation.

### Experimental Section

### EXAMPLE 1

500 g of yellowish raw material, comprising approx. 90% (1S)-3-methylamino-1-thienyl-1-propanol, were distilled at 0.05 mbar and 120° C. evaporation temperature in a molecular distillation apparatus. 420 g (93% yield) of pure-white distillate were obtained as a colorless solid having a content of>99%.

### EXAMPLE 2

500 g of dark-brown raw material, comprising approx. 50% (1S)-3-methylamino-1-thienyl-1-propanol, were distilled at 0.05 mbar and 150° C. evaporation temperature in a molecular distillation apparatus. 221 g (88% yield) of pure-white distillate were obtained as a colorless solid having a content of>99%.

### EXAMPLE 3

500 g of yellowish raw material, comprising approx. 98% (1S)-3-methylamino-1-thienyl-1-propanol, were distilled at 0.05 mbar and 100° C. evaporation temperature in a molecular distillation apparatus. 455 g (93% yield) of pure-white distillate were obtained as a colorless solid having a content of>99%.

1. A method for purifying (1S)-3-methylamino-1-(2-thienyl)-1-propanol by distilling a reaction mixture which comprises between 25 and 99% by weight of (1S)-3-methylamino-1-(2-thienyl)-1-propanol.

2. The method according to claim 1, wherein the distillation is carried out in a pressure range of from 0.0001 to 1 mbar.

3. The method according to claim 2, wherein the distillation is carried out as a molecular distillation.

4 The method according to claim 1, wherein the distillation is carried out at a temperature of from 50 to 250° C.

5. The method according to claim 3, wherein the distillation is carried out at a temperature of from 50 to 250° C.
